(19) United States
(12) Patent Application Publication
(10) Pub. No.: US 2008/0250565 A1
(43) Pub. Date: Oct. 16, 2008

(54) Relocatable Stereotactic Immobilization Apparatus

(75) Inventors: Robert Timmerman, Westlake, TX (US); Lech Papiez, Mooresville, IN (US); Stanley Chien, Zionsville, IN (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: Indiana University Research & Technology Corporation, Indianapolis, IN (US)

(21) Appl. No.: 11/814,407

(22) PCT Filed: Jan. 27, 2006
(86) PCT No.: PCT/US2006/002912

§ 371 Date: Jul. 20, 2007

Related U.S. Application Data
(60) Provisional application No. 60/647,893, filed on Jan. 28, 2005.
(60) Provisional application No. 60/647,920, filed on Jan. 28, 2005.

(51) Int. Cl. A61B 17/66; A61G 7/05

(52) U.S. Cl. 5/621; 5/600

(57) ABSTRACT

A patient immobilization frame is provided that can be secured to a patient table in a radiation therapy facility. The frame includes fiducials integrated into the bottom panel of the frame. The frame is provided with a removable locking mechanism for locking the frame to the patient table. Each side walls of the frame defines a forward cut-out and a mid-side cut-out to accommodate larger girth patients. A panel insert is provided to close the mid-side cut-outs when the additional space is not required for the patient. The frame further includes an abdomen compression device supported by a cross bar that is movably mountable to the frame. The device includes a compression plate configured to contact the patient and a compression mechanism between the cross bar and the compression plate. A pressure transducer may be incorporated between the compression plate and the cross bar.

### REFERENCE TO RELATED APPLICATIONS

This application claims priority to co-pending provisional application No. 60/647,893, entitled “Method for Radiation Therapy Delivery at Varying Source to Target Distances”, filed on Jan. 28, 2005, the disclosure of which is incorporated herein by reference, and to co-pending provisional application No. 60/647,920, entitled “Relocatable Stereotactic Immobilization Apparatus”, filed on Jan. 28, 2005, the disclosure of which is incorporated herein by reference.

### BACKGROUND OF THE INVENTION

The present invention relates to an apparatus used to position and immobilize a patient during a therapeutic or diagnostic treatment. The apparatus is especially useful in procedures that require stereotactic coordination of specific locations in the patient's anatomy with the therapeutic or diagnostic device. In particular, this apparatus is especially well suited to facilitate such procedures on individuals who have wide bodily girth either from large inherent size or from obesity.

Patient positioning systems are used for accurate and reproducible positioning of a patient for radiation therapy, diagnostic imaging, and certain surgical procedures. Stereotactic targeting and immobilization tables support the patient and facilitate precise and accurate guidance for stereotactically directing a variety of therapeutic and diagnostic interventions toward a defined three-dimensional position within the patient's body, including the neck, chest, abdomen, pelvis and proximal thighs.

In a typical radiotherapy procedure, a gantry G (FIG. 1) directs a radiation beam at an iso-center I. The gantry G rotates about a horizontal axis so that the radiation beam is always directed at the iso-center. The iso-center I can be located by the intersection of laser beams generated by wall-mounted laser devices L in the treatment room (FIG. 3). The patient is supported on a couch or table T, as shown in FIG. 2, that can be moved into a position relative to the gantry G and the iso-center I so that the area of treatment can be positioned at the iso-center.

The need for effective patient immobilization for radiation therapy is well-documented. Immobilization reduces normal tissue complication rates and allows increased irradiation of the target tissue. Historically, skin marks have been used to aid in target localization and repositioning. However, skin marks may migrate as they are re-marked and the markings can shift with respect to underlying deeper target tissues. As a consequence, fiducial markings have been placed on patient immobilization frames, since these markings do not smear, fade or migrate. In some procedures, fiducial markings may be matched to skin markings to properly locate and position the target tissue relative to the iso-center.

Patient comfort, the ability of the patient to maintain a position for an extended period of time, reproducibility of the patient's position and anticipated beam orientation must be considered in successful repeat radiotherapy treatments. Patient comfort is essential so that the patient is discouraged from body movement that might be caused by fatigue or pain. Patient movement can invalidate a target localization and expose healthy tissues to unwanted radiation, lead to a diagnostic misinterpretation, or result in mis-targeting of a surgical therapeutic intervention.

To achieve comfortable immobilization, stereotactic body immobilization devices have been developed that support the patient on the couch or table top. In some cases the frames include a body mold that can be stored and re-used in subsequent treatments. The molds are form-fitting and are typically vacuum molded thermoplastic or polyurethane foam molds. In the typical case, the patient is positioned relative to fiducial markings to ensure repeatability over successive treatments.

While current immobilization frames perform well, there are still some aspects that need improvement. For instance, most immobilization frames are designed for the particular treatment system. In other words, the frames are specific to whether the irradiation apparatus is a linear accelerator, a computed tomography apparatus or an MRI device. It is desirable to have an immobilization frame that can be used with a large number of different treatment and diagnostic devices.

Another problem faced by current frame designs is that they cannot be used by patients of large girth. This physical characteristic poses two problems. The first is that most current immobilization frames place the fiducial markings on the side walls of the frame. When a patient of large girth is positioned within the frame, the side walls can bow slightly outward. This displacement of the fiducials disturbs the repeatability of the stereotactic positioning of the patient.

A second problem is that the patient just cannot fit within some of the current frame designs. Many immobilization frames are designed to fit within a limited width dictated by the aperture of the smallest imaging platform, namely MRI, to avoid collision with the imaging unit. In a typical MRI, the aperture is 43 cm. (about 16 in.) so the immobilization frame is necessarily smaller than that dimension (about 40 cm. wide). Many patients seen in diagnostic or therapy clinics, particularly in North America where patients are generally larger, cannot fit into existing frames.

### DESCRIPTION OF THE FIGURES

FIG. 1 is a perspective view of a radiation treatment apparatus.

FIG. 2 is a perspective view of a patient couch or table for use with the treatment apparatus of FIG. 1

FIG. 3 includes a view of a laser device for use in establishing an iso-center and a view of that iso-center.

FIG. 4 is a perspective view of a patient immobilization frame in accordance with one embodiment of the present invention.

FIG. 5 is a rear perspective view of the immobilization frame of FIG. 4.

FIG. 6 is a side elevational view of the immobilization frame of FIGS. 4-5.

FIG. 7 is a top elevational view of the immobilization frame of FIGS. 4-5.

FIG. 8 is a bottom elevational view of the immobilization frame of FIGS. 4-5.

FIG. 9 is an end elevational view of the immobilization frame of FIGS. 4-5.

FIG. 10 is a perspective view of a panel insert for use with the immobilization frame shown in FIGS. 4-9.

FIG. 11 is a perspective view of an immobilization frame according to the present invention with the panel inserts of FIG. 10 in position.

FIGS. 12a-d are perspective and side views of a locking bar assembly for use with the immobilization frame of FIGS. 4-9 in accordance with one aspect of the invention.

FIG. 13 is a top perspective view of a mounting insert forming part of the locking bar assembly shown in FIG. 12.

FIG. 14 is a top elevational view of a locking cam forming part of the locking bar assembly shown in FIG. 12.

FIG. 15 is an enlarged side perspective view of a portion of the immobilization frame of FIGS. 4-9 with the locking bar of FIG. 12 mounted thereto, shown with the frame supported on a patient table.

FIG. 16 is an enlarged side perspective view of the frame and locking bar shown in FIG. 15, with the locking cam inserted into the locking bar.

FIG. 17 is an enlarged view of the locking cam shown in FIG. 16.

FIG. 18 is a side perspective view of the immobilization frame of the present invention with a normally sized patient supported therein.

FIG. 19 is an end perspective view of the patient within the frame of the present invention supported on a patient table and oriented at the iso-center for a treatment apparatus.

FIG. 20 is an end partial cross-sectional view of the immobilization frame of FIGS. 4-9 with an abdominal compression device mounted thereto in accordance with one embodiment of the invention, particularly depicting the relationship of the device to the immobilizing frame and the patient.

FIG. 21 is the side elevational view of the abdominal compression device shown in FIG. 20.

FIG. 22 is a side perspective view of the side of an immobilization frame of an alternative embodiment of the present invention.

FIG. 23 is a top perspective view of the immobilization frame shown in FIG. 22 with an alternative abdominal compression device and a measuring frame mounted on the immobilization frame.

FIG. 24 is an enlarged side view of a hinge component of the abdominal compression device shown in FIG. 23.

FIG. 25 is an enlarged top perspective view of the abdominal compression device shown in FIG. 23.

FIG. 26 is an enlarged side view of a vertical arm of the measuring frame shown in FIG. 23.

### DESCRIPTION OF THE PREFERRED EMBODIMENTS

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

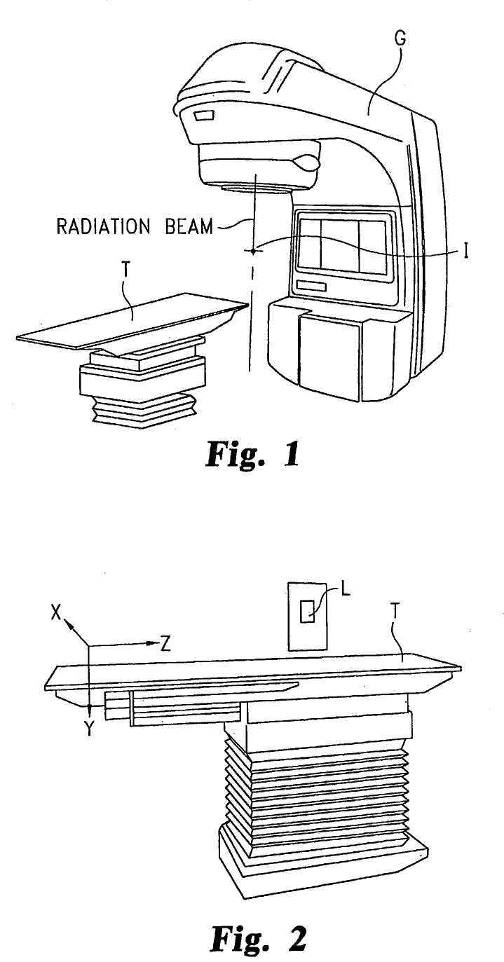

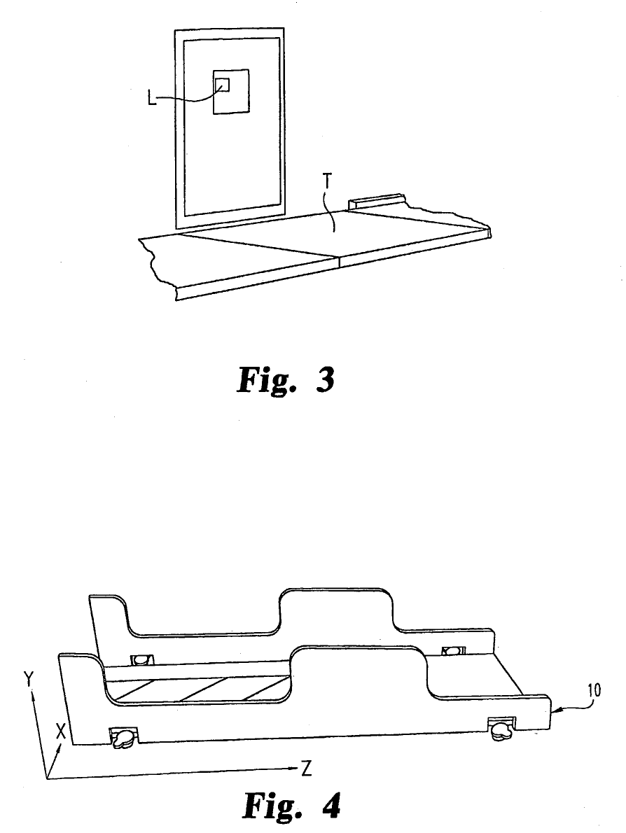

The present invention provides an immobilization frame that can be used with a number of different diagnostic imaging and therapeutic apparatus. A typical system includes a gantry G that rotates around an iso-center I, as shown in FIG. 1, and a patient couch or table T, as shown in FIG. 2. The iso-center can be located by lasers L (FIG. 2-3). The table T establishes a three-dimensional coordinate system (x,y,z) that is used to make measured patient movements or to locate the target tissue. The present invention contemplates an immobilization frame 10 that defines its own three-dimensional coordinate system that can be mapped onto the table coordinate system.

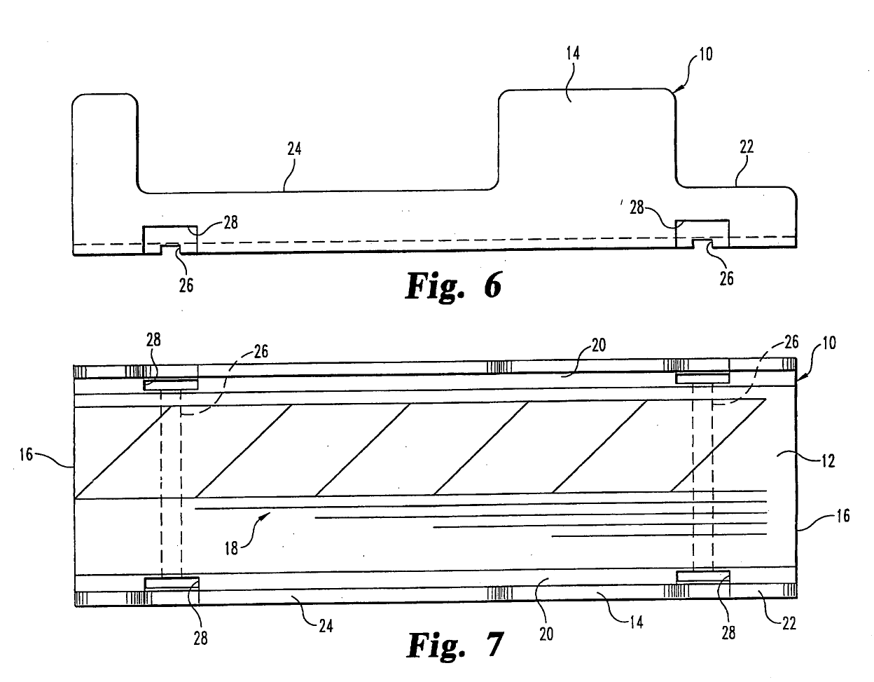

The details of one embodiment of the frame 10 are depicted in FIGS. 5-9. The frame includes a bottom panel 12 that provides the surface on which the patient rests during the irradiation procedure. A pair of opposite side panels 14 project upward from the bottom panel so that the frame defines an open channel. In particular, in the illustrated embodiment, the frame 10 is open at its opposite ends 16.

In one feature of the invention, the bottom panel 12 is provided with an array of fiducial markings 18. Since the fiducials are not on the side panels they cannot be displaced by outward bowing of the panels. Instead, the fiducials are on the bottom panel which will not deform because the bottom panel is supported by the patient table T (FIG. 2). In accordance with the preferred embodiment, the fiducial markings 18 are linear so that the markings can be readily associated with the three-dimensional coordinate system of the frame 10 itself In addition to the fiducial markings 18, the frame can be provided with coordinate markers for use with the room lasers L (FIG. 3) to orient the frame relative to the iso-center I. When the fiducial marks are placed on the bottom panel, the frame can be made as wide as allowed by the MRI, CT or linear accelerator without considering the visibility or accuracy of the side panel on the 3D image.

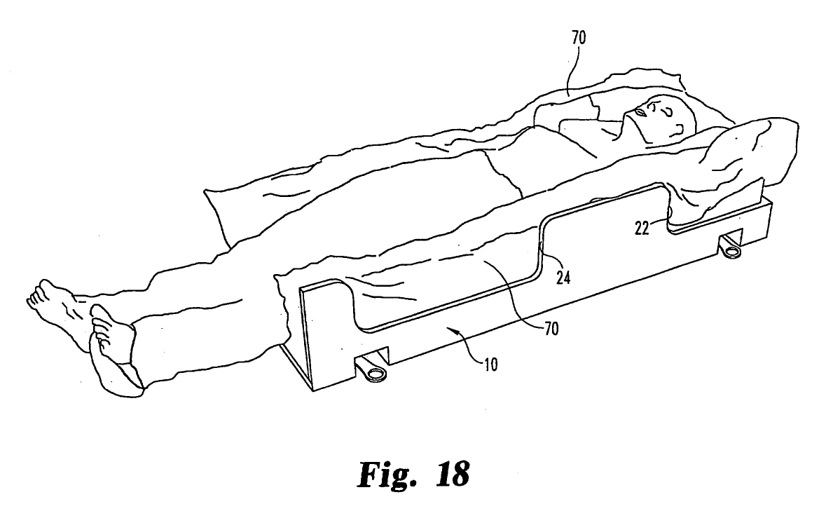

The frame 10 may be provided with angled supports 20 that interface between the bottom panel 12 and the opposite side panels 14. The supports 20 also help establish and maintain the position of the patient within the frame, especially when a positioning mold 71 (FIG. 18) is used.

The side panels 14 include forward cut-outs 22 at the head end of the frame. The forward cut-outs provide clearance around the patient's head and are particularly useful when the patient must position his/her arms in the overhead position shown in FIG. 18.

In one aspect of the invention, the side panels 14 also define mid-side cut-outs 24. Preferably, the mid-side cut-outs 24 extend over about half the length of the frame 10. In addition, the mid-side cut-outs are oriented so that the patient's hips and upper thighs can be aligned with the mid-side cut-outs when the patient is lying in the frame 10, as shown in FIG. 18. Thus, the larger portions of the patients anatomy can extend through the mid-side cut-outs 24 while the patient is positioned with the frame. This aspect of the design directly facilitates the treatment or diagnostic intervention of patients with large girth.

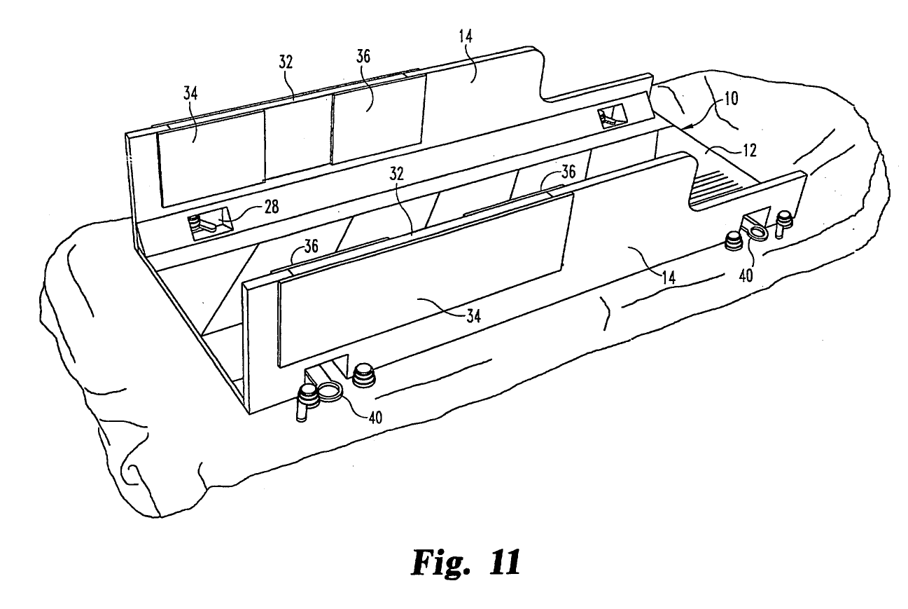

For a normally sized patient, the mid-side cut-outs 24 can be closed with panel inserts 30, shown in FIG. 10. In one embodiment of the invention, the panel inserts includes a center panel 32 that corresponds to the configuration and dimensions of the mid-side cut-outs 24. An outer panel 34 is affixed to the outer face of the center panel 32 and a pair of inner panels 36 are affixed to the inside face and at the ends of the center panel, as show in FIG. 10. The inner and outer panels 34, 36, respectively, cooperate to define an engagement slot 38. As shown in FIG. 11, the panel insert 30 extends into the mid-side cut-outs with portions of the side panel 14 disposed within the engagement slots 38 at the ends of the panel insert.

In one embodiment, the panel insert 30 can be integrally formed as a unitary piece. Gripping handles can be formed into the panel inserts to facilitate placement and removal of the insert from the mid-side cut-outs. While the illustrated embodiment shows the single outer panel 34 disposed on the outside of the frame, a similar construction can be implemented on the inner face of the center panel 32 in lieu of the two inner panels 36. Alternatively, the orientation of the panel insert can be reversed, with the single piece panel 34 extending along the inside of the frame 10. The panel inserts 30 can be configured to support ancillary components of the patient immobilization system, such as straps or body mold portions.

In accordance with one feature of the invention, the underneath surface of the bottom panel 12 is provided with a sliding or friction-resistant coating, such as TEFLON®. This coating allows the frame 10 to slide easily on the patient table T when the patient is positioned within the frame. With this feature, the patient can enter the frame 10 at one end of the patient table T, and then the frame can be manually slid to a pre-determined position on the table to match the frame coordinate system with the table coordinate system. This friction-resistant coating enables the treatment of patients with large girth. Thus, one important feature of the invention is that the frame 10 can be easily slid along the table even when supporting a patient of large overall weight without excessive strain or injury to hospital personnel. Alternatively, the frame 10 may be formed of a material that slides easily on the table surface, such as a carbon-fiber composite.

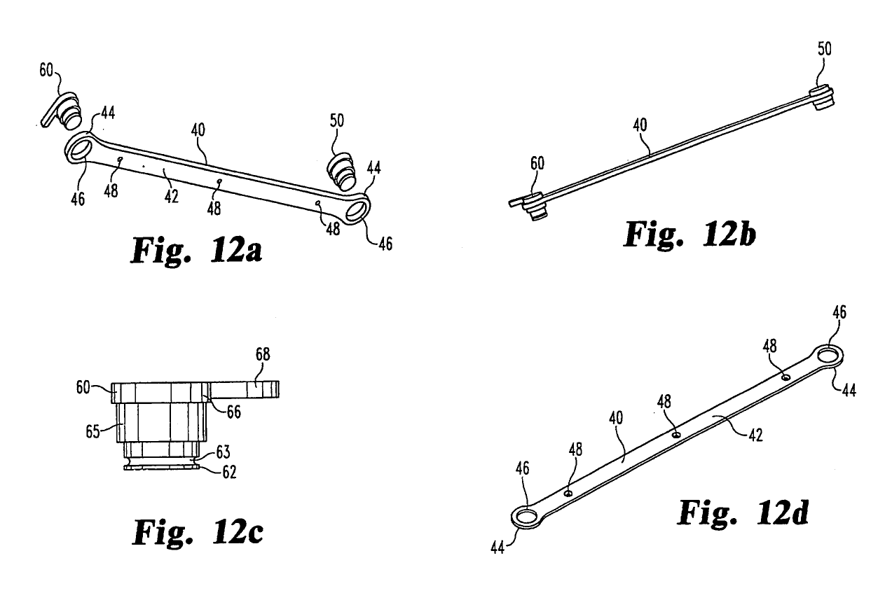

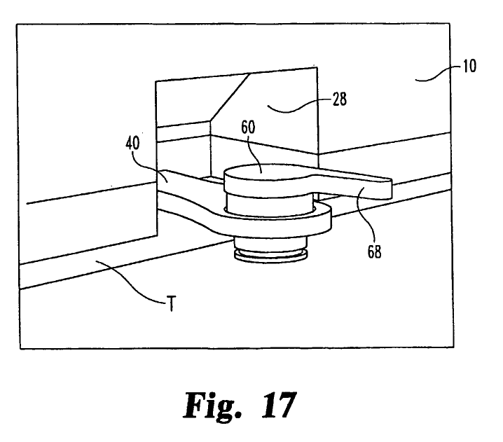

In one important feature of the invention, the frame 10 is also provided with means to rigidly fix the position of the frame relative to the table. In accordance with the invention, a locking bar assembly 40 is provided, as shown in FIGS. 11-17. As shown in FIG. 12(a), the assembly 40 includes a mounting plate 42 that includes enlarged ends 44. Openings 46 are defined in the ends 44 to receive a corresponding mounting insert 50 or locking cam insert 60.

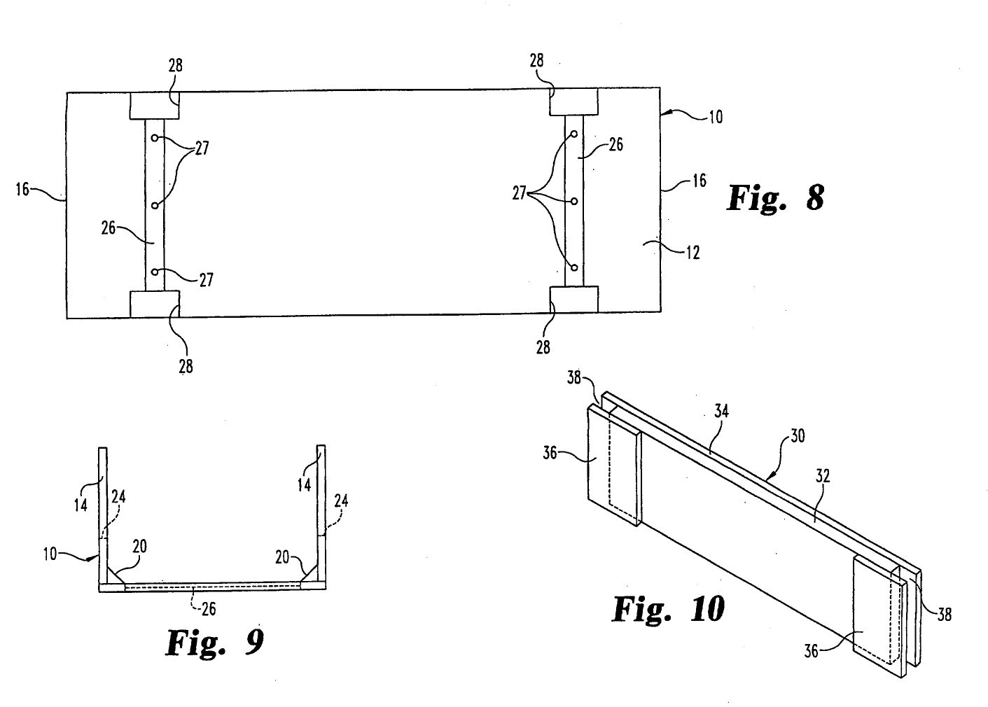

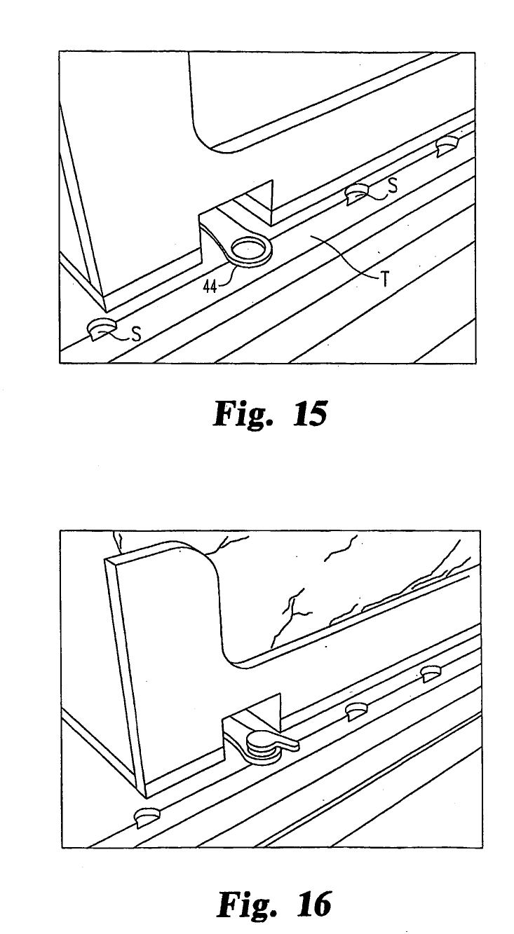

The mounting plate 42 is provided with a plurality of screw holes to fasten the locking bar assembly 40 to the bottom panel 12 of the frame 10. In particular, the bottom panel defines a pair of slots 26 at opposite ends of the frame, as shown in FIGS. 6 and 8. The slots are separated by a pre-determined distance that corresponds to the position of securement recesses on the patient table, such as the recesses S shown in FIG. 15. Thus, as illustrated in FIGS. 15-16, the openings 46 of the locking bar assembly 40 can be positioned over corresponding securement recesses S in opposite sides of the patient table.

Returning to FIG. 8, the bottom panel further defines a plurality of mounting holes 27 in the bottom slots 26. Two mounting plates 42 are attached to the bottom panel 12 within the slots 26 by screws passing through the screw holes 48 into the mounting holes 27. Thus, it can be appreciated that two locking bar assemblies 40 may be rigidly attached to the frame 10, so that when the bar assemblies are rigidly attached to the table T, as described below, the position of the frame 10 is fixed relative to the table T. At this point, then, the coordinates of any point on the frame 10, such as a point on one of the fiducial markings 18, can be translated to the coordinates of the table T, the room coordinates, and ultimately to a position location relative to the iso-center I.

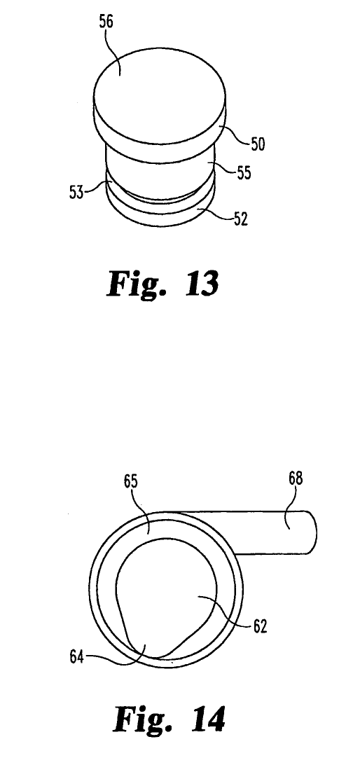

The engagement between the locking bar assemblies 40 and the patient table T is accomplished through the mounting insert 50 and locking cam insert 60. As shown in FIG. 13, the mounting insert 50 includes a bottom disc 52 that is configured to reside at the base of the securement recess S. An engagement groove 53 is provided above the disc to receive a complementary feature within the securement recess S. This engagement between the groove 53 and the complementary recess feature fixes the mounting insert 50 against vertical movement out of the recess.

The mounting insert 50 further includes a post portion 55 that is sized to fit snugly through the opening 46 at one end 44 of the locking bar assembly 40. An enlarged head 56 traps the locking bar assembly beneath the head to prevent removal of the frame from the table once the mounting insert 50 is in its operative position. In the embodiment depicted in FIG. 13, the mounting insert 50 has a cylindrical shape; however, other configurations are contemplated, provided comparable changes are made in the opening 46 at the one end of the locking- bar assembly. Since the mounting insert 50 is intended simply to orient one end of the locking bar, the insert may be semi-circular and engage only half of a circular securement recess S.

The final fixed connection is accomplished by the locking cam insert 60. This insert is configured similarly to the mounting insert in that it is configured to extend through an opening 46 in an opposite end 44 of the locking bar assembly, and to project into and be fixed to a securement recess S in the patient table T. Thus, the insert 60 includes a post portion 65 sized to fit snugly through an opening 46 at the bar end 44, as well as an enlarged head 66. The insert also defines an engagement groove 63 that is engaged by a complementary feature of the securement recess S. However, unlike the other insert 50, the cam insert 60 includes a cam disc 62 that defines an eccentric cam edge 64. A handle 68 is provided on the head 66 to facilitate rotation of the cam insert 60. As the insert 60 is rotated, the eccentric cam edge 64 is disposed within the securement recess to lock the cam insert 60 within the recess. In particular, as the cam edge engages the complementary interior features of the securement recess, the locking cam insert generates a laterally outward force to shift the locking bar assembly laterally relative to the table T. This lateral shift locks the mounting insert 50 in within its table recess S, and continued rotation of the cam insert 60 locks that insert within a securement recess S on the opposite side of the table.

It is contemplated that the mounting insert 50 will be positioned in the ends of the locking bars 40 on one side of the frame, such as the left side. The cam insert 60 is then positioned at the ends of the locking bars on the opposite side of the frame. Thus, when the cam inserts are rotated they uniformly pull the immobilization frame toward that opposite side and effectively lock the mounting inserts 50 within their corresponding table recesses.

In order to facilitate access to and operation of the locking bar assembly, the frame 10 is provided with access cut-outs 28 in the side panels 14 and in the angled supports 20. The access cut-outs are preferably sized so that the corresponding mounting inserts 50 and cam inserts 60 can be positioned within the openings 46 in the locking bar ends 44. In the illustrated embodiment, the openings 46 are oriented just at the opening of the access cut-outs 28. In an alternative embodiment, the side walls 14 of the frame 10 generally coincide with the edge of the table T, so that the access cut-outs 28 are positioned directly above the securement recesses S in edges of the table T.

The locking cam insert provides a means for ready securement of the immobilization frame 10 to the table T by way of the existing securement recesses S. Once the patient is positioned within the frame 10, the low friction surface of the bottom panel 12 allows the frame to be easily slid along the length of the table T until the openings 46 in the ends 44 of the locking bar assembly 40 are aligned with predetermined securement recesses S in the patient table. Mounting inserts can be placed within the bar end openings on one side of the frame. The cam inserts are then placed within the bar end openings on the opposite side of the frame and then rotated to fix the frame 10 to the table T. This rigid securement avoids accidental movement of the frame that might lead to mal-alignment. Moreover, as explained above, this coupling of the frame to the patient table effectively links the frame coordinate system to the table and room coordinate systems. This linking of coordinate systems allows accurate alignment of room mounted devices, such as a linear accelerator beam or a biopsy system, with target tissue in the patient's internal anatomy. Systematic coordinate transformation and error correction algorithms may be used to accomplish this link. This capability can lead to the enabling of a “virtual iso-center” where a variable source-to-target distance is accurately realized.

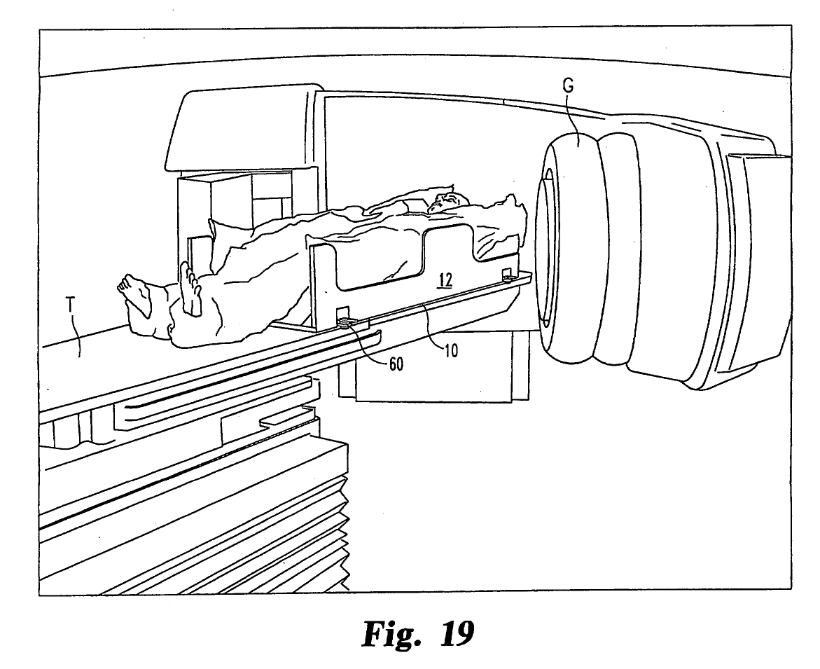

As depicted in FIGS. 18-19, the patient rests within the frame 10, ideally surrounded by a body mold 70 or support cushions or the like, that helps comfortably restrain the patient. It can be appreciated from these figures that a patient of larger girth may require a modified body mold, but at any rate would likely require that the mid-side cut-outs 24 be open.

The frame 10 is preferably formed as a unitary body, with the bottom panel 12, side panels 14 and angled supports 20 integrally formed. In certain embodiments, the frame may be molded from a high density plastic or resin material. The material of the frame must be sufficiently strong so that the side walls 14 do not bend outwardly under pressure from the patient and body mold within the frame.

The mounting plate 42 of the locking bar assembly may be molded directly into the molded frame with appropriate interlocking features to rigidly secure the assembly to the frame. Alternatively, the mounting plate itself may be formed of the same material as the frame so that the plate can be molded as one piece with the bottom panel 12.

In other embodiments, the frame is a carbon fiber frame composed of multiple layer inner and outer skins separated by a honeycomb support structure. The inner and outer skins may be formed of multiple carbon fiber layers glued together by epoxy. The same epoxy may be used to glue the honeycomb structure between the inner and outer skins. One benefit of this material and construction is that the frame may be as lightweight as possible so that it can be easily manipulated in a treatment setting. Of course, any material used to form the frame 10 must not interfere with the operation of the therapeutic or diagnostic equipment.

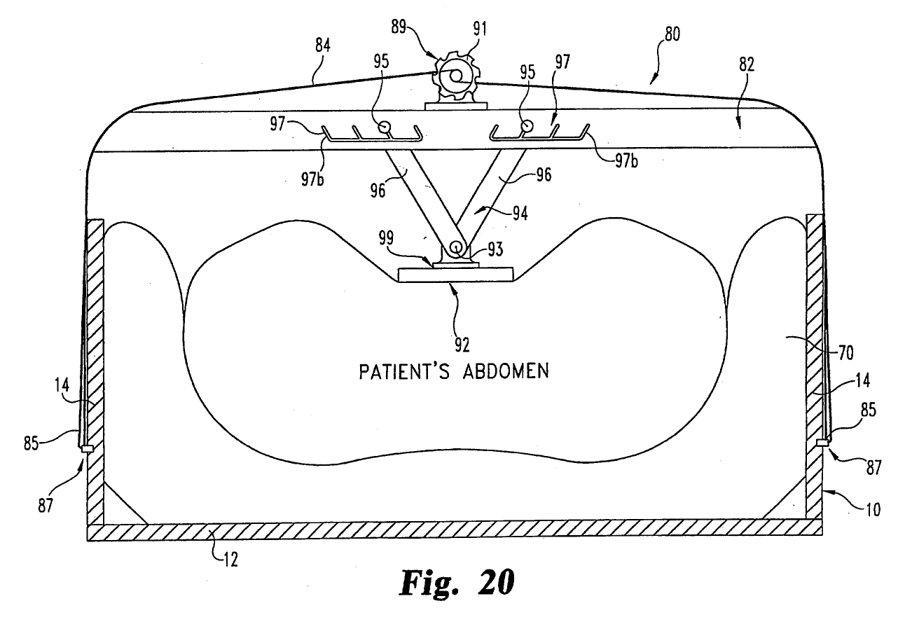

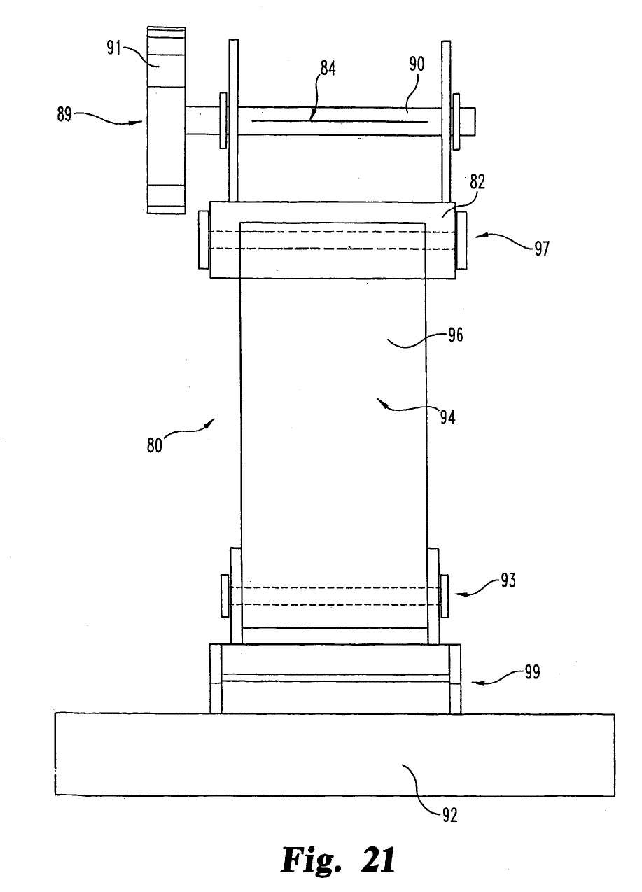

The present invention also contemplates an abdominal compression device for use with the immobilization frame, as depicted in FIGS. 20-21. The device is configured to deliver abdominal compression to restrain the abdomen of the patient for breathing control by altering the fashion in which the lungs expand. In accordance with the invention, the abdominal compression device 80 may be positioned at nearly any point along the length of the immobilization frame depending on the location of the sub-sternal area of the patient.

In accordance with the preferred embodiment, the compression device 80 includes a generally rigid cross beam 82 that is positioned above the patient. Most preferably, the cross beam 82 has a width sufficient to contact the upper edges of the side walls 14 of the immobilization frame 10 to help prevent accidental over-compression by the device. The cross beam is held in a stable position by a strap arrangement or a belt 84 that spans the cross beam and extends down around the side walls 14 of the frame 10, as shown in FIG. 20. The cross beam can include features along its length or particularly at its ends to contain the strap 84 and keep it positioned on top of the cross beam.

The ends of the straps 85 are engaged to the frame 10 through an attachment means 87. In one embodiment, the attachment means can be in the form of a hooked bracket that fits within slots defined in the frame side walls 14. The attachment means 87 is most preferably easily removed from the frame 10. In order to accommodate a wide variety of patient anatomies, the attachment means 87 may provide multiple attachment points along the length of the side walls 14 of the immobilization frame 10.

The patient's abdomen is compressed through a compression plate 92 that is connected to the cross beam 82 through a scissor mechanism 94 attached to the plate at a mounting axle 93. The depth of compression of the device 80 can be adjusted by altering the angle of the scissor mechanism 94. In particular, the mounting ends 95 of the scissor links 96 can be adjustably engaged to the cross beam by a pair of indexing mechanisms 97. The indexing mechanisms 97 provide means for engaging the link ends 95 are different positions along the length of the cross beam. The compression plate is at its greatest depth when the link ends 95 are at the inboard positions 97a of the indexing mechanism 97. Similarly, the plate 92 is at its most shallow position when the link ends are at the outboard positions 97b.

With the compression plate 92 in contact with the patient's abdomen, as shown in FIG. 20, the amount of compression may be adjusted by adjusting the tension in the strap 84, or more specifically by shortening the length of the strap that spans the immobilization frame 10. In accordance with one embodiment of the invention, a strap tightening mechanism 89 is mounted to the top of the cross beam 82. The strap passes through the mechanism, such as through a slotted axle 90. A ratcheting knob 91 is manually rotated to increase the strap tension. A manual tightening mechanism 89 is preferred to minimize the risk of over-tightening the compression device on the patient's abdomen.

The actual pressure generated by the device is measured accurately by an electronic pressure sensor situated within a housing 99 between the compression plate 92 and the scissor mechanism mounting axle 93. In this way, pressure can be consistently and reproducibly applied irrespective of the contents of the patient's stomach on a particular day.

In accordance with the present invention, a stereotactic targeting and immobilization frame is provided that facilitates precise and accurate guidance for stereotactically directing a variety of therapeutic or diagnostic interventions toward a defined three-dimensional position within a patient's body. This inventive frame includes special accommodations for patients with larger body habitus. The frame also includes features for repeat re-positioning, stereotactic coordination both via visual scales and fiducials for a variety of imaging platforms (including plain-films, computed tomography, magnetic resonance imaging, positron emission tomography, and nuclear medicine scans). The frame also incorporates features for accounting for respiratory motion of the patient.

In one feature of the invention, the frame 10 secures rigidly to a linear accelerator or computed tomography couch or patient table by means of locking assemblies. This rigid securement allows the frame coordinate system to be registered to the couch system of coordinates, thereby facilitating automated directed targeting of both therapeutic and diagnostic interventions.

In a further feature, the frame is configured to accept patients having large girth. The frame includes removable panel inserts that leave mid-side cut-outs to allow portions of the patient's body to bulge outside the frame. These removable panels make the frame of the present invention readily usable by larger patients undergoing CT, MRI, PET and other nuclear medicine scans.

The frame 10 provides linear fiducials that provide a straight-forward system for reading the three-dimensional coordinates of any position within the frame itself. This allows accurate registration between PET and nuclear medicine scans and prior CT or MRI imaging scans.

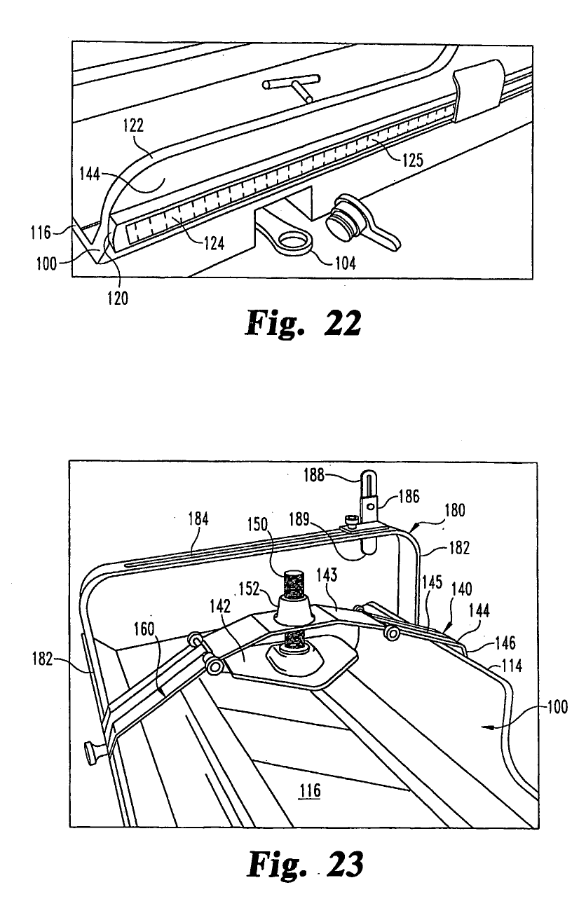

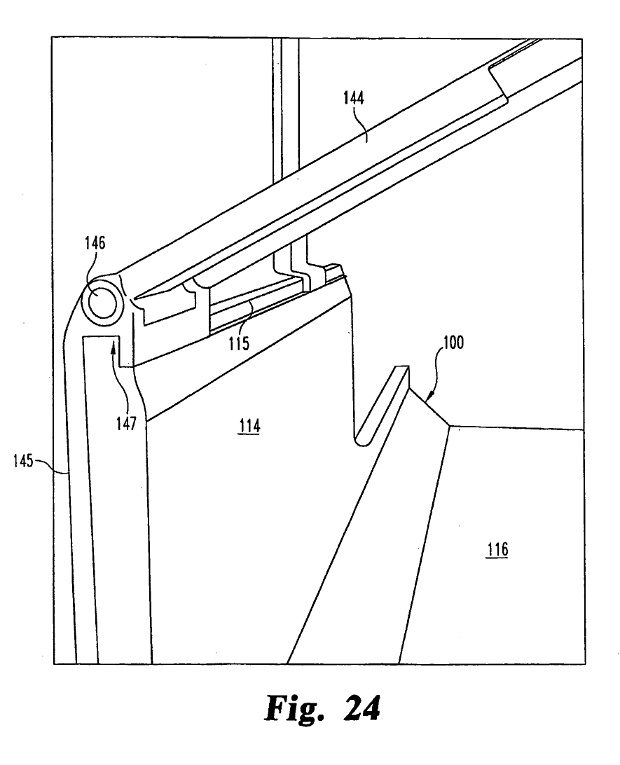

In an alternative embodiment, an immobilization frame 100 may be configured similar to the frame 10 described above. As shown in FIG. 22, the frame 100 includes a bottom panel 116 and side cutouts 122 defined in the side walls 114. In accordance with this embodiment, a mounting bar 124 is affixed to each side immediately above the locking mechanism 104 and extending along substantially the entire length of the frame 100. The mounting bar defines an engagement edge 126 that provides a point of securement for an abdominal compression device 140 and a measuring rig 180 as illustrated in FIG. 23.

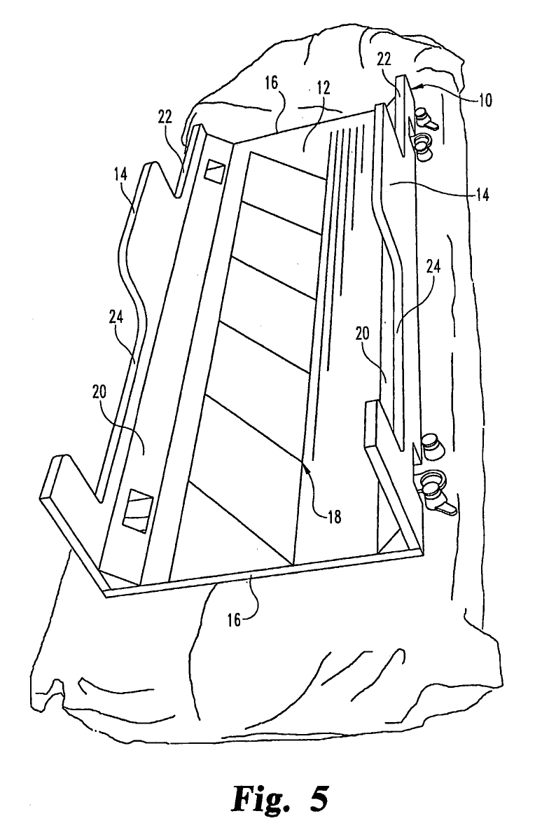

As shown in FIG. 22, the bar includes a measurement scale 125 that provides for measurement along the length of the frame, corresponding to the Z axis shown in FIG. 4. As explained below, the scale 125 permits measurement of the longitudinal location of a measuring rig 180 used to ascertain the location of body features of the patient, or more particularly the position of the target tissue.

Turning to FIG. 23, an alternative embodiment of an abdominal compression device 140 is illustrated. The device 140 includes a compression plate 142 that is configured to comfortably compress the patient's abdomen just below the sternum. The compression plate 142 is supported by a compression screw 150 that passes through a threaded boss 152 on a cross beam 144. The cross beam 144 spans the opposite sides of the frame 100 and more particularly is supported on side walls 114. The cross beam includes a center portion 143 which carries the threaded boss 152 and spanning portions 144 that are pivotably connected to the center portion at hinges 162. The ends of the cross beam 140 are pivotably connected by a pivot 146 to side portions 145 that extend down the side of the immobilization frame 100.

The hinges 162 double as a support for support straps 160. The straps 160 extend over the spanning portions 144 and side portions 145 and include a clip (not shown) at their lower ends for engaging the engagement edges 126 of the mounting bars 124 at the opposite sides of the frame. The hinges 162 may be rotated to tighten the straps to hold the abdomen compression device 140 in solid engagement with the frame. The pivots 146 may incorporate a groove 147 that fits over a top rail 115 on the frame side wall 114 to facilitate movement of the device 140 along the frame while the compression plate 142 is being properly oriented over the patient.

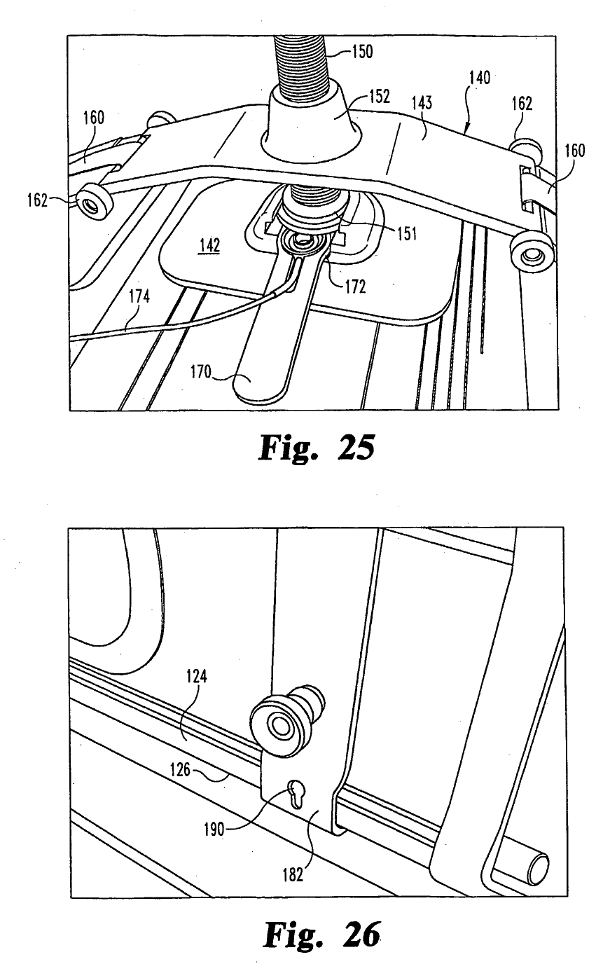

Once the compression plate 142 is properly aligned, the hinges 162 are rotated to tighten the straps 160 to fix the longitudinal location of the abdominal compression device. Then the compression screw 150 is rotated to push the compression plate 142 into the patient's abdomen, as depicted in FIG. 20. The amount of compression may be verified visually; however, in the preferred embodiment, a pressure transducer 172 is introduced between the end 151 of the pressure screw 150 and the compression plate 142, as illustrated in FIG. 25. The transducer 172 is mounted at the end of an introducer 170 that may be used to position the transducer. The transducer includes leads 174 that are fed to a monitor that can be positioned adjacent the table T for easy viewing as the abdominal compression device 140 is manipulated.

The frame 100 of this embodiment further includes a measuring rig 180 that is slidably supported on the side walls 114 of the frame. The rig includes a central beam 184 supported by side beams 182. The side beams are configured at their ends to slidingly mate with the mounting bar 124, as shown in FIG. 26. The side beams 182 include an eyelet 190 that permits visualization of the scale 124 (FIG. 22) to ascertain the longitudinal position of the measuring rig 180 relative to the frame.

The central beam 184 is slotted to receive a position indicator 186. The central beam preferably includes a horizontal scale that defines the lateral location (X axis) of the position indicator 186. The indicator further includes a vertical slide 188 that is mounted to the indicator to slide vertically relative to the central beam. The measuring rig 180 thus provides means for establishing the location of a landmark on the patient in the frame coordinate system (X, Y, Z) by moving the rig until the working end 189 of the vertical slide 188 is aligned with the body feature.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

What is claimed is:

1. A patient immobilization frame for a patient, comprising:
a bottom panel configured to receive the patient positioned thereon; and
opposite side panels, spaced apart a width sufficient to receive the patient positioned therebetween, at least one of said side panels defining at least one cut-out to receive portions of a patient's anatomy of greater girth than said width between said side panels.

2. The patient immobilization frame of claim 1, further comprising a panel insert configured to fit within said at least one cut-out to close said cut-out when a patient is positioned within the frame.

3. The patient immobilization frame of claim 2, wherein said at least one cut-out is a mid-side cut-out disposed in a middle portion of said opposite side panels.

4. The patient immobilization frame of claim 1, wherein each of said opposite side panels includes a forward cut-out extending from a front end of said frame at which the patient's head is located when the patient is received within said frame.

5. The patient immobilization frame of claim 4, wherein each of said opposite side panels further include a mid-side cut-out disposed between said forward cut-out and a rear end of said frame.

6. A patient immobilization frame for a patient, comprising:
a bottom panel configured to receive the patient positioned thereon;
opposite side panels, spaced apart a width sufficient to receive the patient positioned therebetween; and
at least one fiducial incorporated into said bottom panel, said at least one fiducial having a fixed orientation relative to a coordinate system defined in said frame, and said at least one fiducial configured to be observable from outside said frame when a patient is received therein.

7. A patient immobilization frame for supporting a patient on a patient table, the patient table having securement recesses at uniform spaced apart locations along each side of the table, said frame comprising:
a bottom panel configured to receive the patient positioned thereon;
opposite side panels, spaced apart a width sufficient to receive the patient positioned therebetween; and
a locking assembly configured to lock said frame to the patient table, said locking assembly including;
at least two plates projecting from each side of said frame, each of said plates defining an opening therethrough configured to coincide with a corresponding securement recess;
at least two mounting inserts sized to be received through an opening in a corresponding plate at one side of said frame and into a corresponding securement recess at one side of said frame; and
at least two cam inserts sized to be received through an opening in a corresponding plate at the opposite side of said frame and into a corresponding securement recess at the opposite side of said frame, each of said cam inserts defining a cam surface rotatable within the corresponding securement recess to exert a lateral force within the recess to thereby lock said mounting inserts and said cam inserts within their corresponding recesses.

8. The patient immobilization frame of claim 7, wherein said locking assembly further includes:
a pair of locking bars, each sized to span said width and each including said opening at the opposite ends thereof; and
fasteners for securing each of said locking bars to said bottom panel.

9. The patient immobilization frame of claim 8, wherein said bottom panel defines a pair of slots extending across said width, each of said slots sized to receive a corresponding one of said pair of locking bars therein.

10. An abdomen compression device for use with a patient immobilization frame having a bottom panel configured to support the patient thereon and opposite side panels spaced apart a width sufficient to receive the patient therebetween, said compression device comprising:
a cross beam sized to span said width;
a compression plate configured to contact the patient to exert a compression force on the patient's abdomen;
a compression mechanism supported by said cross beam and configured to move said compression plate relative to said cross beam toward the patient; and
a securement mechanism configured for securing said cross beam at variable positions along the side panels of the immobilization frame.

11. The abdomen compression device of claim 10, wherein said compression mechanism includes a scissor mechanism connected between said cross beam and said compression plate.

12. The abdomen compression device of claim 11, wherein said scissor mechanism is connectable to said cross beam at variable positions to vary the distance between said compression plate and said cross beam.

13. The abdomen compression device of claim 10, wherein said compression mechanism includes a threaded boss connected to said cross beam and a screw threadedly extending through said boss and attached to said compression plate.

14. The abdomen compression device of claim 10, wherein said securement mechanism includes a pair of straps connected to said cross beam, one each extending over opposite sides of the immobilization frame, each of said straps including means for engaging the immobilization frame at its side panels.

15. The abdomen compression device of claim 14, wherein said securement mechanism includes means for adjusting the tension in said pair of straps.

16. The abdomen compression device of claim 10, further comprising a pressure transducer disposed between said compression plate and said cross beam.

17. The abdomen compression device of claim 10, wherein said cross beam includes:
a center portion supporting said compression mechanism; and
spanning portions hingedly connected to opposite ends of said center portion, said spanning portions configured to engage the side panels of the immobilization frame.
